(19) United States
(12) Patent Application Publication
(10) Pub. No.: US 2017/0135603 A1
(43) Pub. Date: May 18, 2017

(54) METHODS AND DEVICES FOR POSITIONING OF A MANDIBLE OF A SUBJECT FOR DETERMINING AN OPTIMAL AIRWAY OPENING

(71) Applicant: Kosmo Technologies, Inc., Salt Lake City, UT (US)

(72) Inventors: William H. Hanewinkel, Salt Lake City, UT (US); Mike Gleeson, Salt Lake City, UT (US)

(73) Assignee: Kosmo Technologies, Inc., Salt Lake City, UT (US)

(21) Appl. No.: 15/323,027

(22) PCT Filed: Jun. 30, 2015
(86) PCT No.: PCT/US15/38486

§ 371 Date: Dec. 29, 2016

Related U.S. Application Data
(60) Provisional application No. 62/019,661, filed on Jul. 1, 2014.

(51) Int. Cl. A61B 5/087; A61F 5/56; A61B 5/00

(52) U.S. Cl. CPC A61B 5/087; A61B 5/4818; A61B 5/7275; A61F 5/566; A61B 5/682

(57) ABSTRACT

A point of care device that uses a forced oscillation technique (FOT) and a three dimensional dental device. The point of care device measures the pressure and flow of tidal breathing and reports the results in real time. If the subject's upper airway resistance is high or out of the norm, simply adjust the subject's mandible to see if resistance can be reduced. If it can be reduced by a certain percentage, this will determine if an oral appliance will work for a subject, if that location is comfortable for long term compliance and what the exact location needs to be.

### CROSS-REFERENCE TO RELATED APPLICATION

This application is a national phase entry under 35 U.S.C. §371 of International Patent Application PCT/US2015/038486, filed Jun. 30, 2015, designating the United States of America and published in English as International Patent Publication WO 2016/004004 A1 on Jan. 7, 2016, which claims the benefit of the filing date under Article 8 of the Patent Cooperation Treaty to U.S. Provisional Patent Application Ser. No. 62/019,661, filed Jul. 1, 2014, for “METHODS AND DEVICES FOR POSITIONING OF A MANDIBLE OF A SUBJECT FOR DETERMINING AND MANIPULATING AN AIRWAY OPENING,” the disclosure of each of which is hereby incorporated herein in its entirety by this reference.

### TECHNICAL FIELD

The disclosure relates generally to medical devices and positioning of a mandible of a subject for determining and manipulating an airway opening, for use in treating or screening subjects, for example, breathing disorders involving a subject's airway (e.g., snoring, sleep apnea, upper airway resistance syndrome, etc.). More particularly, but not by way of limitation, the disclosure relates to apparatuses and methods for mandibular manipulation and instant and/or segmented feedback on airway patency using a technique, such as, for example, the noninvasive forced oscillation technique (FOT), impulse oscillometry system (IOS), or other systems and methods for measuring the subject's airway.

### BACKGROUND

In the field of treating the malady of sleep disorders, oral appliances have been shown to be successful in treating mild to moderate cases. The fitting of an oral appliance through, e.g., a dentist has been known for decades to be only in the protrusion of the mandible. Protrusion of the mandible is thought to pull the tongue forward and prevent the tongue from falling back and creating an airway blockage during sleep. The anterior/posterior manipulation of the mandible can also change the shape of the pharyngeal in some individuals to lower the upper airway resistance making the oral appliance more effective. The results of using only protrusion of the mandible with oral appliance therapy are that some subjects respond successfully, some subjects develop side effects such as temporal mandibular joint disorder (TMD), and some subjects do not respond at all. The device and methods described herein create two new medical metrics. The first is a repeatable three-dimensional measurement of the mandible relative to the maxilla, and second is an impedance measurement of the subject's upper airway to relate airway opening to mandible position in real time during mandible adjustment. In most instances this impedance measurement would be conducted using a FOT device but could also be a pressure sensing probe, or shutter/occlusion device as described in international patent application WO 2015/066812 A1, filed Nov. 6, 2014, the disclosure of which is hereby incorporated herein in its entirety by this reference. In practice, the least invasive and most sensitive and accurate pressure measuring technique would be the most desirable.

Generally, obstructive sleep apnea (OSA) is identified through either a Pulmonologist or a Sleep Lab. The diagnosis may be made overnight or otherwise during a time when the subject is asleep. Additionally, preferred treatment measures for OSA were to provide the subject with a continuous positive airway pressure (CPAP) system. In recent years, oral appliance therapy (OAT) has become a known and generally accepted treatment. Many individuals who suffer from breathing disorders do not know it, and a need exists for a point-of-care diagnostic tool to help determine likelihood.

Over the past two decades, OAT has been getting wider recognition as an alternative to positive airway pressure (PAP) devices to treat mild-to-moderate OSA. Oral appliances require no electrical power and are cost effective, quiet, and portable. OAT can be used as a first line treatment, or after patient refusal or intolerance of PAP therapies, or in combination with PAP. Studies show PAP compliance is problematic, ranging from 29% to 83% using their devices less than four hours per night while the hours of sleep could increase to 5.2 hours/night with active intervention. Many subjects prefer OAT over PAP. Side effects of OAT are low, if fitted correctly, but can include excessive salivation as well as mouth and/or teeth discomfort. Adherence rates for OAT are at least equal to PAP when the oral appliance is fitted properly.

Barriers to effective use of OAT by practitioners are twofold. Practitioners receive no feedback regarding airway patency during mandibular titration to ensure not only improved airway patency, but also a comfortable and good fit of the appliance to the subject. There is also no means to adjust oral appliances for anterior, posterior, and vertical positions. Thus, subjects must return to a provider for repeated fitting adjustments. This trial and error procedure becomes time consuming, costly, and potentially discouraging to the subject.

Further, because there is no immediate or quick feedback indicating a successfully improving patient airway patency for the medical practitioner, the fitting of an oral appliance is currently a trial and error method that requires either multiple visits to a subject's dentist to adjust the appliance or utilize a self-adjusting oral appliance that only works in protrusion of the mandible and ignores the vertical position.

Further limitations to the process of fitting an oral appliance include:

No immediate feedback is available to the medical practitioner as to how much protrusion to provide in the mandible.

Multiple trials and visits to the dentist's office for oral appliance adjustments may be required.

Lack of means to adjust both protrusion and vertical of the mandible concurrently while also holding those positions to assess subject comfort with feedback of airway opening.

No means to determine what optimal patency is while manipulating the mandible.

It may be difficult to determine comfort of the subject for long-term oral appliance compliance in combination with optimal airway patency.

Therefore, a need exists for a more efficient methodology to identify optimal airway patency in real-time while having the ability to manipulate the mandible either or both vertically and anterior/posterior. Additionally, it is important to identify a mandibular setting that is comfortable for the subject, for compliancy, and does not place stress on the temporal mandibular joint (TMJ) to cause any temporal mandibular disorders (TMD) that could produce negative long-term effects.

### BRIEF SUMMARY

Described are methods, devices and systems for positioning of a mandible of a subject for determining and manipulating an airway opening and for measuring and adjusting an amount of airflow through an airway of a subject. Such methods may include adjusting a position of the subject's mandible with a dental device (e.g., a dental gauge or mandibular manipulator) in at least one axis of direction and measuring an airway impedance of the subject utilizing a forced oscillation technique. Devices and systems, as disclosed herein, may be utilized to perform such methods.

Disclosed is an apparatus comprising an airflow measurement tube with an open end configured to form an airtight seal against a facial surface of a subject. A dental device is coupled with the airflow measurement tube and configured to position a mandible of the subject with respect to a maxilla of the subject. At least one sensor is coupled with the airflow measurement tube and is configured to measure at least one of an airflow through the airflow measurement tube or a pressure within the airflow measurement tube.

Also disclosed is an airflow measurement tube, comprising an open end configured to form an airtight seal against a facial surface of a subject, a receptacle configured to receive a dental device, and at least one sensor configured to measure at least one of a flowrate through the airflow measurement tube or a pressure within the airflow measurement tube.

Further disclosed is a method of measuring and adjusting an amount of airflow through an airway of a subject, the method comprising positioning the subject's mandible in a first position with a dental device, performing a first airway measurement of the subject's airway with the subject's mandible in the first position, positioning the subject's mandible in a second position with the dental device, performing a second airway measurement of the subject's airway with the subject's mandible in the second position, and comparing the first airway measurement and the second airway measurement.

Yet further disclosed is a system including a manually driven or motor driven dental gauge for measuring and determining mandible position relative to a maxilla in which the means of measuring are performed with a continuous and/or sequential resistive surface that changes resistance to determine position. Micromechanical systems technology may be utilized.

Further disclosed is a method by which manipulating a mandible to create and optimized airway can be utilized to allow medications to be drawn into a respiratory system.

Yet further disclosed is a method by which the upper airway impedance of a subject can be monitored and optimized using frequency oscillation technique in real time while simultaneously using a dental gauge to minutely change a mandible position independently in a vertical as well as a protrusive or at least a single axis of motion. The method may include monitoring and reporting respiratory pressure related to mandibular manipulation in either real time or in a historic manner that relates to mandibular movements. The method may also include visualization of the upper airway of the subject using at least one of MM, computer tomography imaging, ultrasound imaging, endoscopy imaging, or other tools that allow for a visualization of the upper airway. The subject's airway pressure may be measured only during the inspiration portion of the breathing cycle.

Further disclosed is an air tight face mask comprising at least a single axis dental gauge that is surrounded by an acoustically sealed vessel with apertures to allow for a subject to breathe through and in combination with a forced oscillation technique device for measuring flow of tidal breathing and to determine the subject's upper airway impedance in real time or sequential time.

Yet further disclosed is a method utilizing a manually driven or motor driven dental gauge with an ability to lock in place its position ideal to a subject that allows the subject to test comfort before final oral appliance positions are determined and a use of a temporary oral appliance to test subject comfort and efficacy prior to creating a permanent oral appliance.

Further disclosed is a method using a forced oscillation technique with a dental gauge being used in at least one axis to determine if a subject has obstructive sleep apnea, if the subject is a candidate for oral appliance therapy, assists in determining if the subject has other possible apneic root causes, and predicts efficacy of an oral appliance.

Further disclosed is a method of titrating a mandible utilizing multiple modalities such as anterior/posterior gauges, vertical gauges, tongue depressors, and/or other devices that place the mandible in an alternative position in an effort to decrease airway resistance.

Yet further disclosed is a method of utilizing forced oscillation technique and a dental gauge to determine and reduce upper airway impedance and/or increased area of the airway to decrease the impedance of the upper airway to create oral appliances to help in reducing maladies that can be improved by the larger increase in breathing on helping in muscle recovery and performance.

Further disclosed is a manually driven or motor driven dental gauge comprising an airtight sealed mask configured to fit around a mouth and/or nose or configured to be a complete face mask that covers a combined nose and mouth of a subject.

Further disclosed is a method of using an airflow measurement tube in conjunction with a use of a forced oscillation technique device with respiratory pressure reporting in either real time or with sequential positioning of a mandible with respiratory pressure feedback recorded and reported in a historical manner for later analysis.

### BRIEF DESCRIPTION OF THE DRAWINGS

FIG. 1 shows a titration tube applied to a subject.

FIG. 2 is a schematic view of a titration tube combined with frequency oscillation technique components.

FIG. 3 shows a titration tube with mandibular gauge.

FIG. 4 shows a right side view of a titration tube with mandibular gauge.

FIG. 5 illustrates an example chart of airway impedance versus FOT device frequency with an individual in various mandible positions.

FIG. 6 shows an upper portion of the titration tube of FIG. 3.

FIG. 7 is a schematic illustration of a typical operation of a device of the frequency oscillation technique.

### DETAILED DESCRIPTION

This disclosure includes both a method for the practice of manipulating the mandible for an oral appliance fitting while also describing the devices to provide the immediate feedback of the airway opening and the device for manipulating the mandible. Through this method and device, the practitioner now has the means to determine the ideal mandible position that creates the optimal airway opening in both protrusion and vertical when fitting a subject for an oral appliance to treat obstructive sleep apnea. Kosmo Technologies, LLC created this methodology initially using their patented ANDRA GAUGE™. Such a mandibular manipulator is disclosed in U.S. Pat. No. 8,226,407, assigned to Kosmo Technologies, LLC, the disclosure of which is hereby incorporated herein in its entirety by this reference.

The forced oscillation technique (FOT) device is also improved in two ways to minimize its size and weight. One involves the pneumotachograph and the other the acoustic wave emitter. In older designs of pneumotachographs, the pressure sensors were sensitive to humidity, movement of the vinyl tubing, and turbulent airflow. These pneumotachographs utilized electric heaters to prevent condensation and very fine mesh and large diameter screens to create laminar flow. Embodiments of the instant disclosure utilize LDE type pressure sensors from First Sensor AG of Berlin, Germany. Utilizing these high impedance pressure sensors allows us to create a smaller tube/orifice for the pneumotachograph while eliminating the heater and any laminar flow mechanism. First Sensor LDE sensors also create a much improved signal strength and accuracy.

Described is a smaller acoustic wave emitter with the use of “coin type” loudspeaker exciters coupled to a small piston within the breathing tube. Older designs typically use a standard paper cone conical loudspeaker. By using a vented exciter similar to Dayton Audio P/N DAEX19CT-4 and a small plastic piston, the weight and volume of the FOT device can be reduced.

The FOT method can be used to determine the subject's candidacy for oral appliance therapy and to predict effectiveness. This is a major question that insurance companies are trying to answer because there is no way for them to know the effectiveness of an oral appliance on a per-patient basis before incurring the expense of a custom oral appliance plus titration and follow-up sleep study. The FOT technique offers an objective way to determine the treatment plan for all OSA patients. Embodiments of the disclosure offer the potential to answer major questions that third party payers, clinicians, and dentists are trying to answer while fitting oral appliances as a treatment for sleep disorder breathing. The mandible has at least three degrees of freedom and the vertical opening of the mouth is associated with the rotation and sliding of the temporal mandibular joint. There have been multiple scientific research projects to study the effects of a vertical opening with protrusion of the oral cavity with mixed results. A pilot study was conducted that indicated that both vertical and protrusion mandibular manipulation in combination can produce significant airway patency when adjusted with a snoring sound as a feedback mechanism. By creating an immediate feedback metric of the airway while performing mandible titration (A/P and or vertical and or sagittal), the practitioner now has the tools to confidently create an oral appliance that is both effective and comfortable generally in one office visit.

Additionally, by decreasing the upper airway resistance there is an increase in oxygen intake with the use of an OAT fitted with a FOT method. This can directly contribute to increased performance and recovery for athletes, breathing help in elderly subjects, and oxygen uptake in pregnant women. All would benefit from improved oxygen intake with each inspiration.

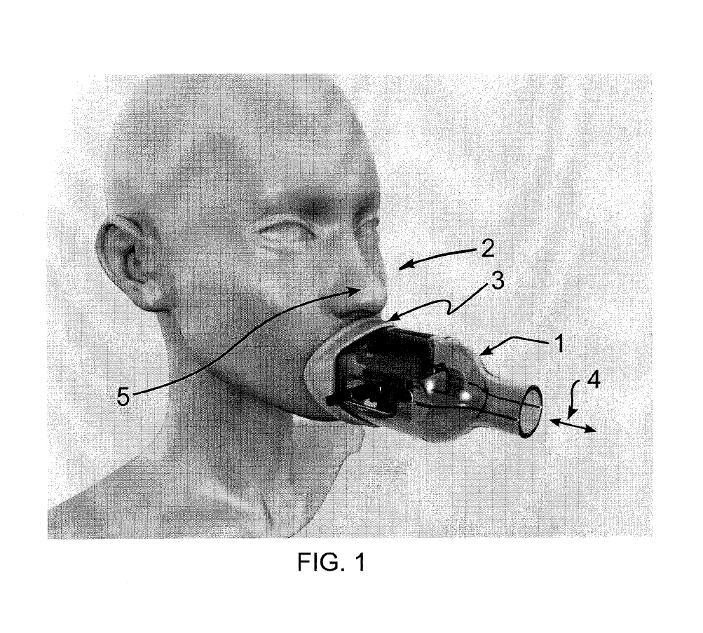

Considering FIG. 1, subject 2 has titration tube 1 pressed against his or her face such that mask 3 ensures an air tight seal between the subject's skin and the mask. The titration tube 1 may also be characterized as an airflow measurement tube which is sized to minimize dead space. (Dead space is defined as the volume of air a subject inspires but never reaches the pulmonary alveoli). In the normal testing method, subject 2 would wear a nasal clamp (not shown) about the subject's nose 5 (e.g., a nasal clamp similar to that manufactured by ARK Therapeutic Services, Inc., 703 Clemson Road, Columbia, S.C. 29229, as part no. 5R). The cylindrical end of the tube 1 would adapt to a similar shape in an airtight manner as it connects to a forced oscillation technique (FOT) device or impulse oscillometry system (IOS). Briefly, FOT superimposes small-amplitude pressure oscillations onto the normal breathing of a subject to determine pressure resistance in the respiratory system. For this application, the upper airway of the subject is of most interest. Although there are a myriad of these devices produced since 1956, such a device might be that produced by MGC Diagnostics, 350 Oak Grove Parkway, St. Paul, Minn. 55127-8599 and known as RESMON™ Pro. Arrow 4 indicates a bidirectional flow of breathing that is performed by the subject through titration tube 1.

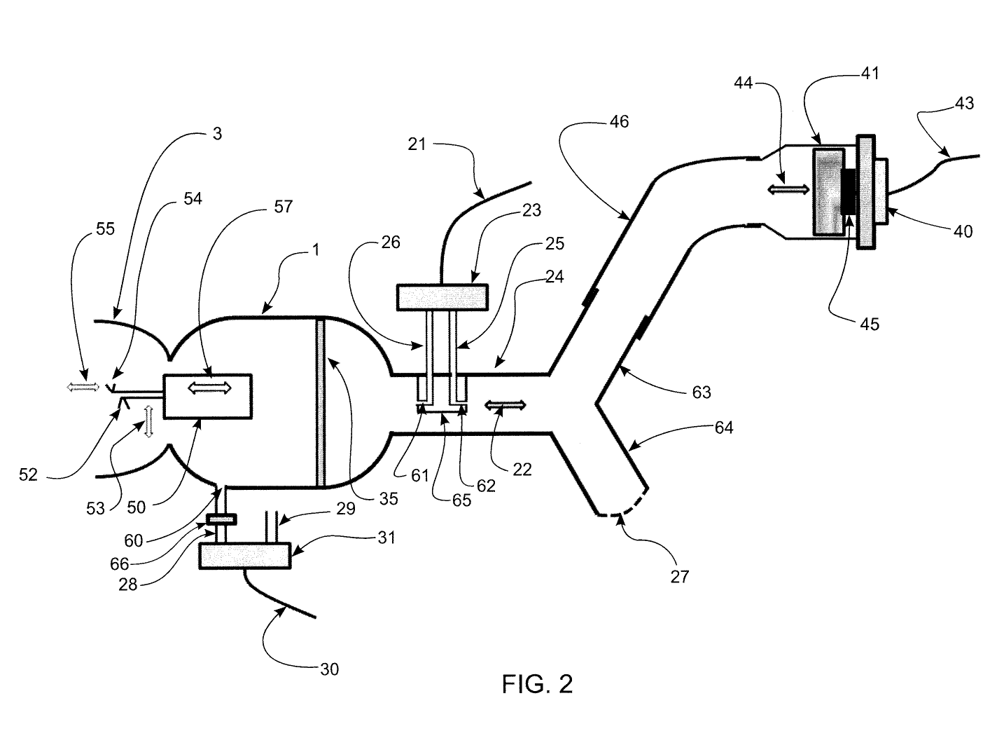

Now considering FIG. 2, there is described a side view functional diagram of the titration tube 1 in combination with the components that offer an embodiment of the disclosure with the use of a frequency oscillation technique. Mask 3 forms an airtight seal to face of subject 2 (FIG. 1). Incisors of subject 2 are placed in the dental gauge attachments 52, 54, as described in U.S. Pat. No. 8,226,407. Gauge attachments 52, 54 interface to the subject's teeth and may be bite arches. Dental gauge 50 has attachments 52, 54 and move in the directions of arrows 53, 55, respectively, for adjusting the mandible of subject 2. Dental gauge 50 is allowed to move relative to titration tube 1 in the direction of arrow 57 to allow facemask 3 to maintain an airtight seal to face of subject 2 when their mandible is adjusted in the anterior/posterior directions, shown by arrow 55. Anti-bacterial filter media 35 (a bidirectional hydrophobic filter media product commonly used in pulmonary devices) is permanently attached within titration tube 1 to help in preventing cross contamination of subject or of pressure sensor 23, tube 24, screen mesh 27, and piston 41 along with its vented exciter 40. Pressure sensor 31 is a differential pressure sensor that senses static pressure in titration tube 1 through vinyl tubing 28 while also measuring atmospheric pressure through tube 29. Pressure sensor 31 is protected from cross contamination by bacterial filter 66 that contains similar material as 35. Multiple conductor cable 30 carries the analog pressure signals back to a digital controller or computer for processing. Pitot Tube 65 is a bidirectional type as described in U.S. Pat. No. 5,379,650 and researched by Kirkness et al., 2011. The pressure of bidirectional flow, arrow 22 generated by subject 2 within cylindrical tube 24 is directed by centerline located orifices 61, 65 and coupled to differential pressure sensor 23 by tubing 25, 26. Multiple conductor cable 21 sends electrical signals to a digital controller or computer to determine the volumetric flow rate of breathing in real time.

Symmetrical wye fitting 63, 64 allows acoustic waves and fresh air to mix upon inspiration while leg 64 and screen 27 allows expired air to escape the apparatus of FIG. 2. Fine mesh screen 27 allows for a bidirectional predetermined impedance flow of air (shown by arrow 22) to the subject 2 while breathing through the FOT device. Coin type audio exciter 40, which is mechanically retained to cylindrical tube 46, creates the FOT pressure waves when coupled with piston 41. Axially driven coil and plate 45, which are part of the exciter 40, coupled with the piston 41 using, for example, an adhesive. Piston 41 moves in a direction of arrow 44. Multiple conductor cable 43 is used to carry an oscillator's electronic signal to exciter 40 to create sinusoidal pressure waves of either a single frequency or composed pseudo random waveform (e.g., sinusoidal pressure waves of 0.5 to 1.0 kPa) within the cylindrical tube 46. Tube 46 can also be a length of flexible tubing which is typically a smooth bore type flexible hose such as manufactured by SMOOTH-BOR PLASTICS, 23322 Del Lago Drive, Laguna Hills, Calif. 92653.

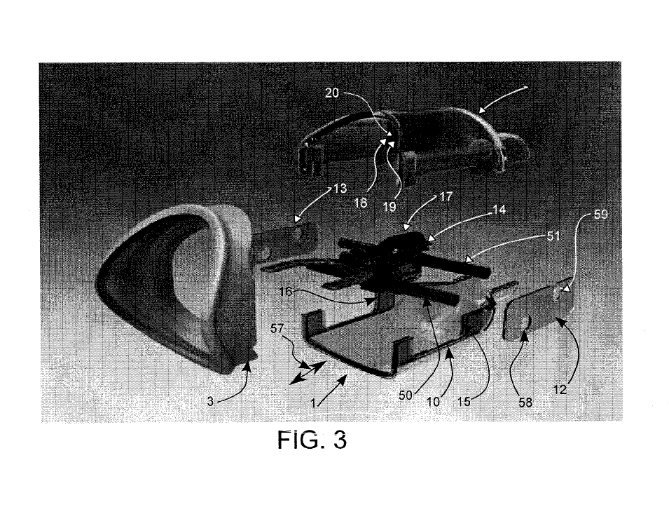

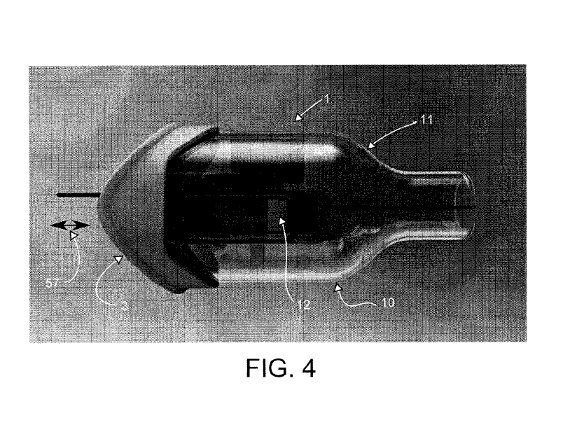

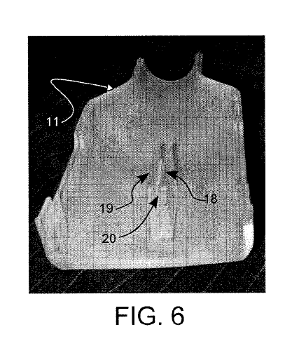

Now considering FIGS. 3, 4, and 6, the titration tube 1 comprises two polymer shells 10, 11 that come together forming an airtight seal around sliding members 12, 13, dental gauge 14, and is enclosed on one end by mask 3. Dental gauge 14 is described in its entirety by U.S. Pat. No. 8,226,407. The subject's upper and lower incisors engage the dental gauge 14 as described in U.S. Pat. No. 8,226,407. Sliding members 12, 13 engage grooves 15 and 16 and have apertures that match the dental gauge handles 50, 51 such that they can pass through apertures 58, 59, respectively. On the opposite side of titration tube 1, sliding member 13 functions symmetrically in an identical manner. Sliding members 12, 13 form an acoustic seal with titration tube 1 while allowing the dental gauge 14 to move in the directions of arrow 57 so that the subject's mandible can move relative to the mask 3 without losing the airtight seal at the subject's face. Stiffener 17 of gauge 14 slides within the gap 20 formed by bifurcated structures 18, 19 that are an integral part of shell 11. As described previously, gauge 14 slides relative to titration tube 1. The before-mentioned bifurcated structures 18, 19 keep the gauge 14 centered within titration tube 1 while static or sliding (FIG. 6).

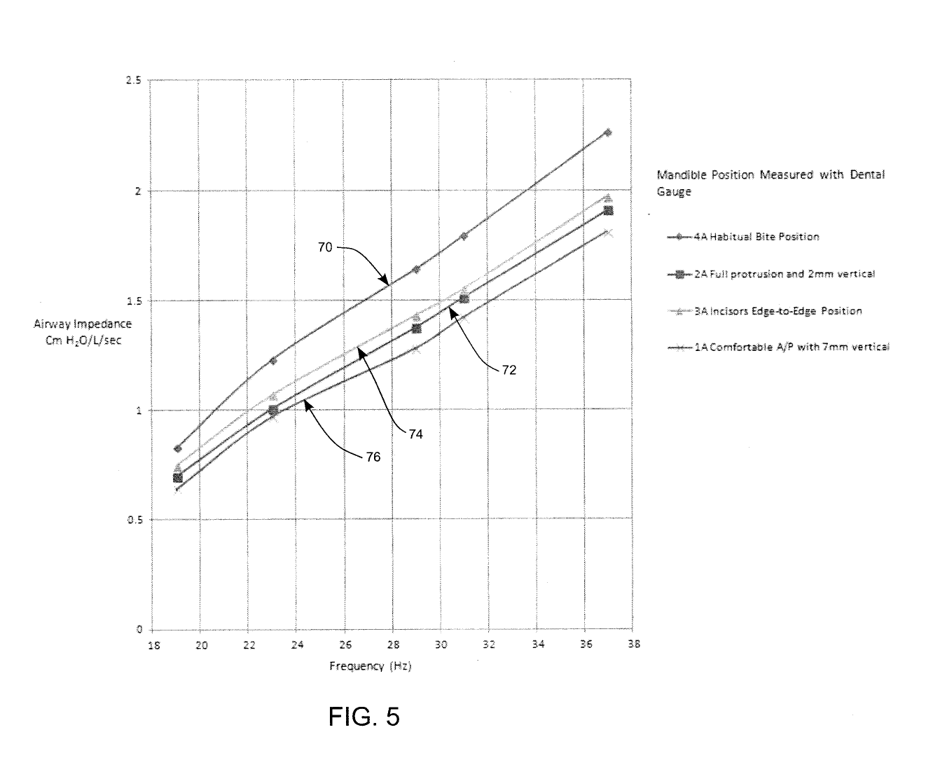

Focusing on FIG. 5, the chart is an example indication of how airway impedance can change using titration tube 1 and an FOT device. In this case, the individual does not have any respiratory maladies. The FOT device used is manufactured by MGC Diagnostics, 350 Oak Grove Parkway, St. Paul, Minn. 55127-8599. However, any FOT device reading real time results for this example would report similar respiratory impedance values for the given mandible positions. Respiratory impedance readings for FIG. 5 were taken at sinusoidal frequencies of 19, 23, 29, 33, and 37 Hz, respectively. In this experiment, the individual is fitted to a prototype of the titration tube 1, with his incisors engaged into the dental gauge, and a soft foam rubber mask creates the airtight seal between the titration tube 1 to his face. Chart line 70 reports the respiratory impedance of the individual whilst his mandible is positioned in a habitual bite position. Chart line 72 reports the respiratory impedance of the individual whilst his mandible is fully protruded anteriorly and the vertical position is 2 mm, which is the minimum vertical position of the dental gauge. The spacing between lines 70 and 72 represents a respiratory reduction in impedance of approximately 16% from the habitual bite position. However, the full protrusion position of chart line 72 is not comfortable so two other more comfortable positions are attempted. Chart lines 74 and 76 represent mandible positions that are more comfortable to the individual. Chart line 74 reports respiratory impedance of the individual whilst his mandible is positioned with a vertical of 2 mm and the protrusion of the incisors edge-to-edge. While line 74 represents a more comfortable mandible position, the respiratory impedance is reduced and is approximately 13% lower than the habitual mandible setting. Chart line 76 reports respiratory impedance of the individual whilst his mandible is set at a comfortable anterior position and 7 mm vertical. Chart line 76 reports not only a comfortable mandible position for the individual but an approximately 20% reduction in respiratory impedance over the habitual mandible position. Empirical data suggest that a vertical position of between about six millimeters (6 mm) and about nine millimeters (9 mm) may reduce (e.g., minimize) airway impedance relative to other vertical positions in a majority of subjects. Therefore, a vertical position of between about six millimeters and about nine millimeters may be used as a starting vertical position to reduce (e.g., minimize) the number of test positions required to determine a position with suitable levels of airway impedance and comfort.

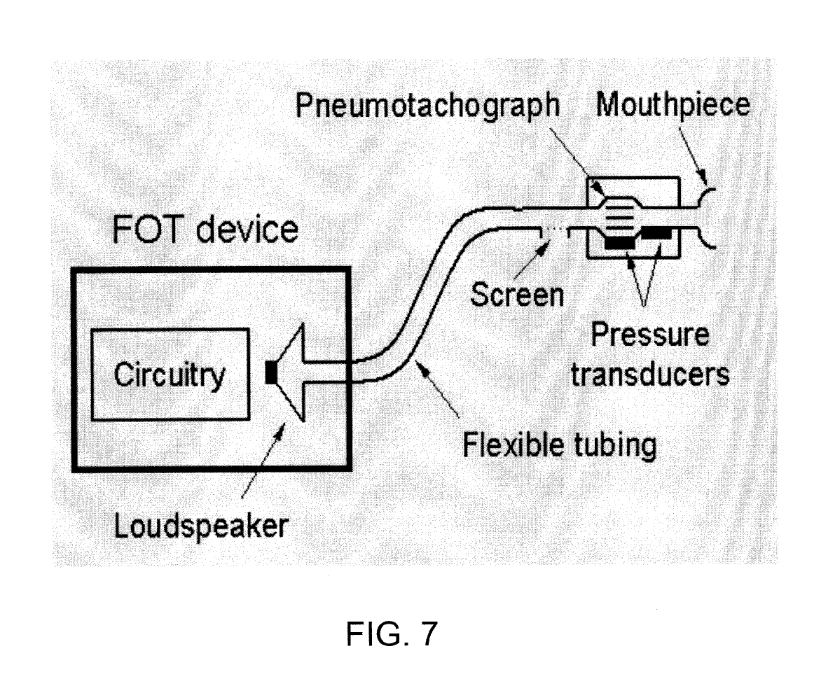

FIG. 7 is a reference figure to explain the typical operation of a device of the frequency oscillation technique. Of note is the large loudspeaker (e.g., similar to or larger to than TS-W161, 60, loudspeakers made by Pioneer of Des Moines, Iowa). Since some embodiments of this disclosure do not need low frequency response of the loudspeaker (e.g., around 5 Hz), the device can use the smaller coin size speaker exciter as described earlier. Also, the Fleisch pneumotachograph, which is used for recording the subject's tidal breathing, is generally required to be more complicated in typical FOT devices. Because of the older pressure sensor technology used, the pneumotachograph requires a heater and five minute warm-up period, along with means of creating laminar flow of the air. With micromechanical systems (MEMS) technology, both heater and laminar flow screens are eliminated in embodiments of the disclosure to measure a subject's tidal breathing flow rate. Either a venturi flow metering device or a pitot tube can be utilized as described in U.S. Pat. No. 5,088,332 with the pitot tube being the preferred method as described by Kirkness, et al., 2011.

Once being apprised of the instant systems and devices for mandibular manipulation and feedback on airway patency, one of ordinary skill in the art will be readily able to make and assemble such systems and devices.

1. An apparatus comprising:
an airflow measurement tube with an open end configured to form an airtight seal with an airway of a subject;
a dental device coupled with the airflow measurement tube and configured to position a mandible of the subject with respect to a maxilla of the subject; and
at least one sensor coupled with the airflow measurement tube and configured to measure at least one of an airflow through the airflow measurement tube or a pressure within the airflow measurement tube.

2. The apparatus of claim 1, wherein the dental device is configured to alter the position of the mandible with respect to the maxilla in at least two orthogonal directions.

3. The apparatus of claim 2, wherein the dental device is configured to lock in place to maintain the position of the mandible with respect to the maxilla.

4. The apparatus of claim 2, wherein the at least two orthogonal directions define an anterior/posterior position of the mandible with respect to the maxilla and a vertical position of the mandible with respect to the maxilla.

5. The apparatus of claim 1, wherein the dental device is configured to adjust and position the mandible with respect to the maxilla in the anterior/posterior direction, the vertical direction, and the sagittal direction.

6. The apparatus of claim 1, wherein the first end of the airflow measurement tube is configured to form an airtight seal simultaneously around the subject's mouth and the subject's nasal passages.

7. The apparatus of claim 1, wherein the at least one sensor comprises a differential pressure sensor.

8. The apparatus of claim 1, wherein the at least one sensor comprises a pitot tube.

9. The apparatus of claim 1, further comprising an acoustic wave emitter coupled with the airflow measurement tube and configured to induce pressure waves within the airflow measurement tube.

10. The apparatus of claim 9, wherein the acoustic wave emitter is configured to induce sinusoidal pressure waves of a single frequency within the airflow measurement tube.

11. The apparatus of claim 9, wherein the acoustic wave emitter is configured to induce pressure waves with a composed pseudo random waveform within the airflow measurement tube.

12. The apparatus of claim 9, wherein the acoustic wave emitter is coupled to a piston positioned in an opening of the airflow measurement tube.

13. An airflow measurement tube, comprising:
an open end configured to be positioned against a facial surface of a subject;
a receptacle configured to receive a dental device; and
at least one sensor configured to measure at least one of a flowrate through the airflow measurement tube or a pressure within the airflow measurement tube.

14. The airflow measurement tube of claim 13, further comprising openings configured to enable adjustment handles of the dental device to extend outside of the airflow measurement tube.

15. The airflow measurement tube of claim 14, wherein the airflow measurement tube is configured to form an airtight seal around the adjustment handles of the dental device while enabling movement of the dental device.

16. The airflow measurement tube of claim 15, further comprising sliding members positioned within the openings and forming an airtight seal between the openings and the adjustment handles of the dental device.

17. A method of measuring and adjusting an amount of airflow through an airway of a subject, the method comprising:
positioning the subject's mandible in a first position with a dental device;
performing a first airway measurement of the subject's airway with the subject's mandible in the first position;
positioning the subject's mandible in a second position with the dental device;
performing a second airway measurement of the subject's airway with the subject's mandible in the second position; and
comparing the first airway measurement and the second airway measurement.

18. The method of claim 17, wherein positioning the subject's mandible in the first position comprises positioning the subject's mandible at a vertical distance of between about six millimeters (6 mm) and about nine millimeters (9 mm) from the subject's maxilla.

19. The method of claim 17, wherein performing a first airway measurement and performing a second airway measurement comprise measuring a first airway impedance and a second airway impedance using a forced oscillation technique or an impulse oscillometry system.

20. The method of claim 17, wherein performing a first airway measurement and performing a second airway measurement comprise measuring a first airway impedance and a second airway impedance using an occlusion-type airway measurement technique.
